SEQUENCE LISTING (4 sequences)

SEQ ID NO: 1 - protein, 101 aa, artificial sequence
  Monomer of spider silk DP-1A analog protein
GAGRGGQGAGAAAAAAGGAGQGGYGGLGSQGAGRGGLGGQGAGAAAAAAAGGAGQGGLGSQGAGQGAGAAAAAAGGAGQGGYGGLGSQGAGQGGYGGLGSQ

SEQ ID NO: 2 - protein, 101 aa, artificial sequence
  Monomer of spider silk DP-1B.9 analog protein
GAGQGGYGGLGSQGAGRGGLGGQGAGAAAAAAAGGAGQGGLGSQGAGQGAGAAAAAAGGAGQGGYGGLGSQGAGRGGQGAGAAAAAAGGAGQGGYGGLGSQ

SEQ ID NO: 3 - protein, 101 aa, artificial sequence
  Monomer of spider silk DP-1B.16 analog protein
SQGAGQGGYGGLGSQGAGRGGLGGQGAGAAAAAAAGGAGQGGLGSQGAGQGAGAAAAAAGGAGQGGYGGLGSQGAGRGGQGAGAAAAAAGGAGQGGYGGLG

SEQ ID NO: 4 - protein, 119 aa, artificial sequence
  Monomer of spider silk DP-2A analog protein
GPSGPGSAAAAAAGPGQQGPGGYGPGQQGPGGYGPGQQGPSGPGSAAAAAAAAGPGGYGPGQQGPGGYGPGQQGPSGPGSAAAAAAAAAGPGGYGPGQQGPGGYGPGQQGPGGYGPGQQ

(19) United States
(12) Patent Application Publication
(10) Pub. No.: US 2004/0132978 A1
(43) Pub. Date: Jul. 8, 2004

(54) Method for purifying and recovering silk proteins in soluble form and uses thereof

(76) Inventors: Stephen R. Fahnestock, Wilmington, DE (US); Thomas M. Schultz, Randolph, NJ (US)

Correspondence Address: POTTER ANDERSON & CORROON LLP, ATTN: KATHLEEN W. GEIGER, ESQ., P.O. BOX 951, WILMINGTON, DE, 19899-0951, US

(21) Appl. No.: 10/704,337

(22) Filed: Nov. 7, 2003

Related U.S. Application Data
(60) Provisional application No. 60/425,617, filed on Nov. 12, 2002.

(51) Int. Cl. A61K 38/17

(52) U.S. Cl. 530/353

(57) ABSTRACT

A method for purifying and recovering silk proteins in water-soluble form is described. The method is based upon precipitation of the silk protein at a temperature below room temperature, which results in a protein pellet that redissolves in water without the addition of harsh chemicals. When the precipitation is done at room temperature, the resulting protein pellet cannot be redissolved in water. Applications for the water-soluble silk proteins in cosmetics, skin care, hair care, hair coloring products, and for pigment coating and wound healing bandages are described.

This application claims the benefit of U.S. Provisional Application NO.: 60/425,617, filed Nov. 12, 2002.

### FIELD OF THE INVENTION

The invention relates to the field of purification and recovery of proteins from a sample. More specifically, the invention relates to a method for the purification and recovery of recombinant silk proteins in water-soluble form using precipitation at low temperature.

### BACKGROUND OF THE INVENTION

Silks are some of the strongest natural fibers known, rivaling high performance synthetic fibers in mechanical properties. Strong natural fibers with high tensile strength and elasticity are useful for many applications, including high strength fibers for textile applications and composite materials, such as parachutes, sails and body armor. Additionally, silk proteins have low immunogenic and allergenic potential, making them suitable for medical applications such as wound sutures, membranes, surfaces for cultivated cells, and as a scaffold for artificial organs. Silk proteins self-assemble in solution (Winkler et al., Int. J. Biol. Macromol. 24:265-270 (1999)), making them useful in applications requiring film formation and surface coating, such as skin and hair care products, particle coating, and in wound dressings.

Silks are produced by over 30,000 species of spiders and by many insects particularly in the order Lepidoptera (Foelix, R. F. (1992) Biology of Spiders, Harvard University Press Cambridge, Mass.). Few of these silks have been studied in detail. The cocoon silk of the domesticated silkworm Bombyx mori and the dragline silk of the orb-weaving spider Nephila clavipes are among the best characterized. Although the structural proteins from the cocoon silk and the dragline silk are quite different from each other in their primary amino acid sequences, they share remarkable similarities in many aspects. They are extremely glycine and alanine-rich proteins. Fibroin, a structural protein of the cocoon silk, contains 42.9% glycine and 30% alanine. Spidroin 1, a major component of the dragline silk, contains 37.1% glycine and 21.1% alanine. They are also highly repetitive proteins. The conserved crystalline domains in the heavy chain of the fibroin and a stretch of polyalanine in Spidroin 1, are repeated numerous times throughout entire molecules. These crystalline domains are surrounded by larger non-repetitive amorphous domains in every 1 to 2 kilobases in the heavy chain of Fibroin, and by shorter repeated GXG amorphous domains in tandem in Spidroin 1. They are also shear sensitive due to their high copy number of the crystalline domains. During fiber spinning, the crystalline repeats are able to form anti-parallel β-pleated sheets, so that silk protein is turned into semi-crystalline fiber with amorphous flexible chains reinforced by strong and stiff crystals (Kaplan et al., (1997) in Protein-Based Materials, McGrath, K., and Kaplan, D. Eds, Birkhauser, Boston, pp 104-131).

Traditional silk production from silkworm involves growing mulberry leaves, raising silkworm, harvesting cocoons, and processing silk fibers. It is labor intensive and time consuming and therefore prohibitively expensive. Additionally, silkworm silk proteins are very insoluble in aqueous solutions and can only be dissolved using harsh solvents.

Similarly, the production of spider silk from cultivated spiders is limited by the small amounts of silk produced, making commercially useful quantities of silk protein unattainable at a reasonable cost. Additionally, multiple forms of spider silks are produced simultaneously by any given spider. The resulting mixture has less application than a single isolated silk because the different spider-silk proteins have different properties and, due to solubility problems, are not easily separated by methods based on their physical characteristics. Hence the prospect of producing commercial quantities of spider silk proteins from natural sources is not a practical one. Moreover, considerable difficulty has been encountered in attempting to solubilize and purify natural spider silk proteins while retaining the molecular-weight integrity of the fiber. The silk fibers are insoluble except in very harsh agents such as LiSCN, LiClO4, or 88% (vol/vol) formic acid. Once dissolved, the protein precipitates if dialyzed or if diluted with typical buffers.

Recombinant DNA technology has been used to produce silk proteins. Ohshima et al. (Proc. Natl. Acad. Sci. USA, 74:5363-5367 (1977)) report the cloning of the silk fibroin gene complete with flanking sequences of the silkworm Bombyx mori into E. coli. Petty-Saphon et al. (EP 0230702) disclose the recombinant production of silk fibroin and silk sericin from a variety of hosts including E. coli, Sacchromyces cerevisiae, Pseudomonas sp., Rhodopseudomonas sp., Bacillus sp., and Strepomyces sp. These recombinant silkworm silk proteins are expressed in insoluble form and can only be dissolved using harsh solvents.

The production of recombinant spider silk proteins is also known. Xu et al. (Proc. Natl. Acad. Sci. U.S.A., 87:7120-7124 (1990)) report the determination of the sequence for a portion of the repetitive sequence of a dragline spider silk protein, Spidroin 1, from the spider Nephila clavipes, based on a partial cDNA clone. Hinman and Lewis (J. Biol. Chem. 267:19320-19324 (1992)) report the sequence of a partial cDNA clone encoding a portion of the repeating sequence of a second fibroin protein, Spidroin 2, from dragline silk of Nephila clavipes. Lewis et al. (U.S. Pat. Nos. 5,728,810 and 5,989,894) disclose the expression of spider silk proteins including protein fragments and variants of Nephila clavipes from transformed E. coli. Two distinct proteins were independently identified and cloned and were distinguished as silk protein 1 (Spidroin 1) and silk protein 2 (Spidroin 2). cDNA clones encoding minor ampullate spider silk proteins and the expression thereof is described by Lewis et al. (U.S. Pat. Nos. 5,733,771 and 5,756,677). Lewis et al. (U.S. Pat. No. 5,994,099) describe the cloning of cDNA encoding the flagelliform silk protein from an orb-web spinning spider. Fahnestock (U.S. Pat. No. 6,268,169) describes novel spider silk analog proteins derived from the amino acid consensus sequence of repeating units found in the natural spider dragline of Nephila clavipes. The synthetic spider dragline was produced from E. coli, Bacillus subtilis, and Pichia pastoris recombinant expression systems. Spider silk proteins and analog proteins have also been expressed in plants (Yang, WO 01/90389, and Scheller et al., DE 10113781). The water solubility of the spider silk proteins expressed in plants is reported to be much higher than that of the bacterially expressed proteins, as they are readily soluble in aqueous buffers (Scheller et al. DE 10113781). Additionally, spider silk proteins have been expressed in mammalian cells (Lazaris et al., Science 295:472-476 (2002)) and in transgenic animals (Clark et al. in published US Patent Application No. 2001/0042255 and Karatzas et al. in patent application WO 99/47661)

Recombinant spider silk proteins are expressed in soluble form in bacterial hosts and in both soluble and insoluble forms in yeasts. However, the recombinant spider silk proteins that are expressed in soluble form become insoluble upon purification and are extremely difficult to resolubilize after drying or precipitation (Arcidiacono et al., Macromolecules 35:1262-1266 (2002)), limiting applications which require processing of the proteins into different types of fibers, films or coatings. Winkler et al. (Int. J. Biol. Macromol. 24:265-270 (1999)) report that recombinant spider silk proteins rapidly self-assemble upon purification to form insoluble microfibrils. Fahnestock (Rev. Mol. Biotechnol. 74:105-119 (2000)) also reports that once spider silk analog proteins were precipitated, they could only be redissolved in denaturing solvents, such as aqueous quanidine hydrochloride or hexafluoroisopropanol.

Methods have been developed to solubilize silk proteins and to keep them in solution during purification. For example, Stedronsky in U.S. Pat. No. 5,760,004 describes a method to enhance the water solubility of repetitive polymers, including those that mimic the properties of the silk protein of Bombyx mori, by reacting small polar molecular weight reactants with available functionalities on the protein. The resulting product is water-soluble, but the chemical modifications may adversely alter the properties of the silk protein. Additionally, Lombardi et al. (U.S. Pat. No. 5,245,012) describe a method for solubilizing spider silk proteins that comprises dissolving the protein in a solution that consists of a 50-50 volume ratio of propionate and hydrochloric acid. Basel et al. (WO 97/08315) report that recombinant spider silk protein can be solubilized by the use of ultrasound, but only if the protein was not washed and completely dried. They further report that denaturants, such as urea, guanidine hydrochloride, hexafluoroisopropanol, lithium thiocyanate and lithium bromide, will keep the spider silk protein from reprecipitating prior to spinning into silk fibers. Because recombinant spider silk proteins become insoluble during or after purification, chromatographic purification methods have been developed that use organic acids, such as formic acid and propionic acid or denaturing agents, such as guanidine hydrochloride and urea, to keep the spider silk protein in solution (Mello et al., WO 01/53333, Arcidiacono et al, Macromolecules 35:1262-1266 (2002), Arcidiacono et al., Appl. Microbiol. Biotechnol. 49:31-38 (1998), and Lewis et al., Protein Expression and Purification 7:400-406 (1996)). In all these disclosures, harsh chemical reagents are used to solubilize the spider silk proteins and to keep them in solution. The use of these harsh chemicals is not compatible with many applications of silk proteins, for example, personal care products and medical applications.

Capello et al. (U.S. Pat. No. 5,235,041) teach a method for the purification of structurally ordered recombinant protein polymers, such as silk proteins. This method utilizes the low solubility characteristics to isolate and purify silk proteins. The expressed protein product is rendered insoluble in the host cells by heating or cooling. The cells are lysed and the protein produced is separated from other components of the lysate and then extracted to remove contaminants while remaining in insoluble form. However, the resulting purified protein is not water soluble.

Lazaris et al. (Science 295:472-476 (2002)) describe the production of soluble recombinant spider silk protein produced in mammalian cells. The Supplementary Material for this article (Science 295 Number 5554, Issue of Jan. 18, 2002, 472) describes a purification method utilizing an ammonium sulfate precipitation step at 4° C. The precipitated protein was readily redissolved in phosphate-buffered saline, unlike recombinant spider silk proteins produced in E. coli or yeasts, which could only be redissolved in strong denaturing solvents after precipitation at room temperature (Fahnestock, Rev. Mol. Biotechnol. 74:105-119 (2000)). The solubility of the spider silk proteins produced in mammalian cells was attributed to the presence of the COOH-terminus in these proteins which makes them more hydrophilic. These COOH-terminal amino acids are absent in spider silk proteins expressed in microbial hosts.

The use of precipitation by the addition of salts, organic solvents or water-soluble polymers to purify and recover proteins is well known in the art (Scopes, Protein Purification Principles and Practice, 2^{nd} edition, Springer-Verlag, NY, 1982; Chapter 3, Deutscher, Guide to Protein Purification, Methods in Enzymology, Vol. 182, Academic Press, NY, 1990; Chapter 22, and Doonan, Protein Purification Protocols, Methods in Molecular Biology, Vol. 59, Humana Press, Totowa, N.J., 1996; Chapter 13). However, these disclosures do not teach that silk proteins precipitated at a temperature below room temperature are readily redissolved in water while those precipitated at room temperature can not be redissolved in water without the use of harsh chemical reagents.

Silk proteins have found application in personal care products, pigment coating, and in bandages to promote wound healing. The use of silk protein peptides, formed from the hydrolysis of silk proteins, and their derivatives in cosmetics and hair care products has been described (Oshika et al. U.S. Pat. No. 5,747,015, Terada et al. JP 27186, and Kuroda et al. JP 309816). In these disclosures, silk protein hydrolysates were used because of the low solubility of the intact silk proteins. While providing some beneficial coating effect, the silk protein peptides are not as effective as the intact proteins. Ritter et al. (DE 3139438) describe the use of colloidal silk protein as an additive in hair care products. However, the colloidal silk protein is not as effective in film-forming and coating for hair treatment as a soluble silk protein. Philippe et al. in U.S. Pat. No. 6,280,747 describe the use of natural or recombinant spider silk proteins in cosmetic and dermatological compositions such as hair care, skin care, make-up, and sunscreen products. However, the spider silk proteins described in that disclosure are not water soluble. Therefore, the beneficial effects of the self-assembly and coating properties of the spider silk proteins are not realized.

Hasegawa et al. in U.S. Pat. No. 6,296,860 describe the use of N-acylated silk protein amino acids to coat pigments and extender pigments. Again, the isolated amino acids are not as effective in coating particles as the soluble intact silk protein. Otoi et al. in U.S. Pat. No. 4,325,741 describe the use of silkworm silk protein to coat pigments. However, harsh solvents were required to dissolve the silk protein.

Silk proteins have also found application in wound dressing material as a healing promoter. Pickart in U.S. Pat. No. 5,382,431 describes the use of an enzyme digest of silk protein in a method to accelerate the healing of topical wounds. The digested silk protein is not as effective as a water-soluble silk protein. Tsubouchi in U.S. Pat. No. 6,175,053 describes a wound dressing material containing a healing agent which comprises an amorphous film containing silkworm silk and sericin as the main component. Harsh solvents were required to dissolve the silk protein.

The need exists for silk proteins that remain soluble after purification and that can be redissolved in aqueous solutions after precipitation without the use of harsh chemical reagents. These water-soluble silk proteins have applications in personal care products such as cosmetics, skin care, hair care and hair coloring; in coating of particles, such as pigments; and in bandages to promote wound healing.

### SUMMARY OF THE INVENTION

The invention provides a method for the purification of water-soluble silk protein from a sample containing water-soluble silk protein comprising;

a) providing a sample comprising silk protein in the presence of contaminating proteins, wherein at least a portion of the silk protein is water soluble;

b) adjusting the pH of the sample of a) to an acidic pH;

c) heating the adjusted sample of b) to a temperature of at least about 55° C.;

d) removing debris from the sample of c);

e) lowering the temperature of the heated sample of (d) to below about 20° C.;

f) adding an effective amount of precipitating agent to the cooled sample of e) for a time sufficient to allow silk protein to precipitate, wherein the precipitated silk protein may be redissolved in an aqueous solution.

In an alternate embodiment the invention provides a method for the purification of water-soluble silk protein from a host cell containing water-soluble silk protein comprising:

a) providing a host cell comprising silk protein, a portion of which is water soluble;

b) disrupting the host cell to release the silk protein and produce a crude silk extract;

c) adjusting the pH of the crude silk extract to an acidic pH;

d) heating the adjusted extract of c) to a temperature of at least about 55° C.;

e) removing cell debris from the extract of d);

f) lowering the temperature of the heated extract of (e) to below about 20° C.;

g) adding an effective amount of precipitating agent to the cooled extract of f) for a time sufficient to allow silk protein to precipitate, wherein the precipitated silk protein may be redissolved in an aqueous solution.

A suitable precipitation agent for use in the invention is ammonium sulfate.

Suitable host cells for the production of the silk or silk-like protein of the invention includes but is not limited to prokaryotic cells, yeasts, fungi, algae, green plants, and mammalian cells.

A preferred silk for use in the invention is spider dragline silk having the general formula:

[AGQGGYGGLGXQGAGRGGLGGQGAGAnGG]z

wherein X=S, G or N; n=0-7 and z=1-75, and wherein the value of z determines the number of repeats in the variant protein and wherein the formula encompasses variations selected from the group consisting of:

(a) when n=0, the sequence encompassing AGRGGLGGQGAGAnGG is deleted;

(b) deletions other than the poly-alanine sequence, limited by the value of n will encompass integral multiples of three consecutive residues;

(c) the deletion of GYG in any repeat is accompanied by deletion of GRG in the same repeat; and

(d) where a first repeat where n=0 is deleted, the first repeat is preceded by a second repeat where n=6; and wherein the full-length protein is encoded by a gene or genes and wherein said gene or genes are not endogenous to the Nephila clavipes genome;

or having the general formula:

[GPGGYGPGQQGPGGYGPGQQGPGGYGPGQQGPSGPGSAn]z

wherein n=6-10 and z=1-75 and wherein, excluding the poly-alanine sequence, individual repeats differ from the consensus repeat sequence by deletions of integral multiples of five consecutive residues consisting of one or both of the pentapeptide sequences GPGGY or GPGQQ and wherein the full-length protein is encoded by a gene or genes and wherein the gene or genes are not endogenous to the Nephila clavipes genome.

Additionally the invention provides a cosmetic, skin care, or hair care or hair coloring composition comprising: an effective amount of a water-soluble silk protein purified by the method of the invention.

Alternatively the invention provides a pigment or cosmetic particle coated with an effective amount of a water-soluble silk protein purified by the method of the invention.

In an alternate embodiment the invention provides a film or fiber coated with an effective amount of a water-soluble silk protein purified by the method of the invention.

Similarly the invention provides a wound healing bandage coated with an effective amount of a water soluble silk protein purified by the method of the invention.

In another embodiment the invention provides a water-borne lacquer for use in a nail varnish comprising an effective amount of a water-soluble silk protein purified by the method of the invention.

In an alternate embodiment the invention provides a water-soluble silk protein purified by the method of the invention which has been derivatized with a functional group selected from the group consisting of amines, oxanes, cyanates, carboxylic acid esters, silicone copolyols, siloxane esters, quaternized amine aliphatics, urethanes, polyacrylamides, dicarboxylic acid esters, and halogenated esters.

### BRIEF DESCRIPTION OF THE SEQUENCE DESCRIPTIONS

The invention can be more fully understood from the following detailed description and the accompanying sequence descriptions, which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NO:1 is the amino acid sequence of the monomer of the spider silk DP-1A analog protein.

SEQ ID NO:2 is the amino acid sequence of the monomer of the spider silk DP-1B.9 analog protein.

SEQ ID NO:3 is the amino acid sequence of the monomer of the spider silk DP-1B.16 analog protein.

SEQ ID NO:4 is the amino acid sequence of the monomer of the spider silk DP-2A analog protein.

### DETAILED DESCRIPTION OF THE INVENTION

There is a need, described herein, for water-soluble silk and silk-like proteins that can be redissolved in aqueous solution without the use of harsh chemicals after precipitation with a precipitaing agent. Applicants have solved the stated problem by unexpectedly discovering that recombinant silk proteins that are purified by precipitation at a temperature below room temperature are readily redissolved in water. The self-assembly characteristics of these water-soluble silk proteins makes them ideally suited for film-forming and coating applications such as cosmetics, skin care, hair care and hair coloring products; in coating of particles, such as pigments; and in bandages to promote wound healing.

The following definitions are used herein and should be referred to for interpretation of the claims and the specification.

“Nucleic acid” refers to a molecule which can be single stranded or double stranded, composed of monomers (nucleotides) containing a sugar, phosphate and either a purine or pyrimidine. In bacteria, lower eukaryotes, and in higher animals and plants, “deoxyribonucleic acid” (DNA) refers to the genetic material while “ribonucleic acid” (RNA) is involved in the translation of the information from DNA into proteins.

The terms “polypeptide” and “protein” are used interchangeably.

The term “peptide” is used to describe a subunit of a polypeptide or protein formed by hydrolysis.

“Gene” refers to a nucleic acid fragment that effects the production of a specific protein, including regulatory sequences preceding (5″ non-coding sequences) and following (3″ non-coding sequences) the coding sequence.

“Codon” refers to a unit of three nucleotides that encodes a single amino acid.

As used herein, “transformation” is the acquisition of new genes in a cell by the incorporation of nucleic acid.

The term “expression” as used herein is intended to mean the transcription and translation to gene product from a gene coding for the sequence of the gene product. In the expression, a DNA chain coding for the sequence of gene product is first transcribed to a complementary RNA which is often a messenger RNA and, then, the thus transcribed messenger RNA is translated into the above-mentioned gene product if the gene product is a protein.

The terms “silk variant protein” and “silk analog protein” are used interchangeably herein to refer to a designed protein, the amino acid sequence of which is based on repetitive sequence motifs and variations thereof that are found in a known natural silk protein.

The term “full length variant protein” will refer to any silk variant protein encoded by a synthetic gene, which has been constructed by the assembly and polymerization of a DNA monomer.

The term “DNA monomer” will refer to a DNA fragment consisting of between 300 and 400 bp which encodes one or more repeating amino acid sequences of a silk variant protein.

The term “peptide monomer”, or “polypeptide monomer” will refer to the amino acid sequence encoded by a DNA monomer.

The term “DP-1 analog” will refer to any spider silk variant derived from the amino acid sequence of the natural Protein 1 (Spidroin 1) of Nephila clavipes.

The term “DP-2 analog” will refer to any spider silk variant derived from the amino acid sequence of the natural Protein 2 (Spidroin 2) of Nephila clavipes.

As used herein the following abbreviations will be used to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Asparagine or aspartic acid | Asx | B |
| Cysteine | Cys | C |
| Glutamine | Gln | Q |
| Glutamine acid | Glu | E |
| Glutamine or glutamic acid | Glx | Z |
| Glycine | Gly | G |
| Histidine | His | H |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The present invention comprises a method for the purification and recovery of water-soluble silk proteins that can be redissolved in water after precipitation from a sample that contains a water-soluble silk protein. A water-soluble silk protein is herein defined as a recombinant silk protein or analog, having a glycine-rich sequence, herein referred to as the soft segment, alternating with an oligomer of polyalanine, herein referred to as the hard segment, in which at least about 20% of the soft segment is composed of glycine and about 70-100% of the hard segment is composed of alanine. The length of the hard segment is between about 5 to 100 amino acids, while the length of the soft segment is between about 5 to 300 amino acids. At least some portion of the silk protein is initially present in soluble form, but when precipitated it would not normally redissolve in aqueous solution without the addition of harsh chemicals such as acids or denaturants. The present invention provides a method for purifying and recovering silk proteins by precipitation from the sample wherein the precipitated protein redissolves in aqueous solution without the addition of harsh chemicals. Although the discussion herein is limited to silk proteins, one skilled in the art will recognize that the method of the present invention is also applicable to a wide variety of protein polymers having long stretches of repeating units, where the repeating units are generally of from about 3 to 50 amino acids. Such proteins include, but are not limited to, members of the collagen family, keratin, elastin, fibronectin, laminin and other fibrous proteins, and variants thereof.

The recombinant silk proteins include, but are not limited to, spider silk proteins and spider silk analog proteins. Analog silk proteins are herein defined as polypeptides that imitate the repeating units of amino acids of natural silk proteins. The terms “analog silk protein” and “silk variant protein” are herein used interchangeably. For example, the silk protein may be recombinant dragline spider silk protein, specifically, Spidroin 1 or Spidroin 2, or variants thereof, originating from the major ampullate gland of Nephila clavipes, as described by Lewis et al. in U.S. Pat. Nos. 5,728,810 and 5,989,894, incorporated herein by reference. Additionally, the silk protein may be the recombinant spider silk proteins originating from the minor ampullate gland of Nephila clavipes, or variants thereof, as described by Lewis in U.S. Pat. Nos. 5,733,771 and 5,756,677, incorporated herein by reference. The silk protein may also be the recombinant silk protein originating from the flagelliform gland of Nephila clavipes, or variants thereof, as described by Lewis in U.S. Pat. No. 5,994,099, incorporated herein by reference. The silk protein may also be one or more of those described by Lewis et al. in Protein Expression and Purification 7:400-406 (1996), or by Prince et al., in Biochemistry 34:10879-10885 (1995), or by Winkler et al. in Int. J. Biol. Macromol. 24:265-270 (1999). Moreover, the silk protein may be a variant designed to possess certain beneficial properties. For example, the silk protein variant may be designed to have increased elasticity by elongating the glycine-rich (soft) segment, as described in patent applications WO 9116351 and EP 452,925, both of which are incorporated herein by reference. Conversely, it is possible to reduce the elasticity of the silk protein by shortening the glycine-rich segment. Similarly, the replacement of the glycine residues with serine residues results in a less rigid silk protein.

The preferred water-soluble silk proteins of the present invention are spider silk analog proteins, as described by Fahnestock in U.S. Pat. No. 6,268,169, incorporated herein by reference. That disclosure describes analog proteins of the natural dragline spider silk Spidroin 1 (DP-1) and Spidroin 2 (DP-2) proteins of Nephila calvipes. Two analogs of DP-1 were designed and designated DP-1A and DP-1B. DP-1A is composed of a tandemly repeated 101-amino acid sequence. The 101-amino acid “peptide monomer”, given as SEQ ID NO:1, comprises four repeats which have different patterns that reflect the variation of the individual repeating units of DP-1 from the consensus sequence. This 101-amino acid long peptide monomer (SEQ ID NO:1) was repeated from 1 to 16 times in a series of analog proteins. DP-1B was designed by reordering the four repeats within the monomer of DP-1A. Two sets of genes using different codons were designed to produce DP-1B, specifically DP-1B.9 and DP-1B.16. The resulting amino acid monomer sequences are given as SEQ ID NO:2 for DP-1B.9 and SEQ ID NO:3 for DP-1B.16. The sequence of DP-1B matches the natural sequence of Spidroin 1 more closely over a more extended segment than does DP-1A. The DP-1 amino acid sequences and similar analogs may be represented by the following consensus repeat formula:

[AGQGGYGGLGXQGAGRGGLGGQGAGAnGG]z (1)

wherein X=S, G or N; n=0-7 and z=1-75, and wherein the value of z determines the number of repeats in the variant protein. The formula encompasses variations selected from the group consisting of: (a) when n=0, the sequence encompassing AGRGGLGGQGAGAnGG is deleted; (b) deletions other than the poly-alanine sequence, limited by the value of n will encompass integral multiples of three consecutive residues; (c) the deletion of GYG in any repeat is accompanied by deletion of GRG in the same repeat; and (d) where a first repeat where n=0 is deleted, the first repeat is preceded by a second repeat where n=6; and wherein the full-length protein is encoded by a gene or genes and wherein said gene or genes are not endogenous to the Nephila clavipes genome.

Synthetic analogs of DP-2 were designed to mimic both the repeating consensus sequence of the natural protein and the pattern of variation among individual repeats of Spidroin 2. The analog DP-2A, given as SEQ ID NO:4, is composed of a tandemly repeated 119-amino acid sequence. This 119-amino acid “peptide monomer” comprises three repeats which have different patterns. This 119-amino acid long peptide monomer was repeated from 1 to 16 times in a series of analog proteins. The spider silk analog protein DP-2A is the most preferred water-soluble silk protein of the present invention. The DP-2 amino acid sequence and similar analogs may be represented by the following consensus repeat formula:

[GPGGYGPGQQGPGGYGPGQQGPGGYGPGQQGPSGPGSAn]z (2)

wherein n=6-10 and z=1-75 and wherein, excluding the poly-alanine sequence, individual repeats differ from the consensus repeat sequence by deletions of integral multiples of five consecutive residues consisting of one or both of the pentapeptide sequences GPGGY or GPGQQ and wherein the full-length protein is encoded by a gene or genes and wherein the gene or genes are not endogenous to the Nephila clavipes genome.

The water-soluble silk proteins may be prepared by transformed prokaryotic or eukaryotic systems including bacterial, yeast, plant, and mammalian systems, using standard recombinant DNA techniques. These recombinant DNA techniques are described by Sambrook, J., Fritsch, E. F. and Maniatis, T. Molecular Cloning: A Laboratory Manual, Second Edition, Cold Spring Harbor Laboratory Press, Cold Spring Harbor (1989), and Clark et al., Plant Molecular Biology, A Laboratory Manual, Springer-Verlag, Berlin, Heideiberg, 1997, both of which are incorporated herein by reference.

For example, the water-soluble silk proteins may be expressed in plants such as Arabidopsis or soy somatic embryos as described by Yang in patent application WO 01/90389, incorporated herein by reference. Additionally, silk analog proteins may be expressed in the endoplasmic reticulum of the leaves or tubers of transgenic tobacco and potato plants, as described in patent application DE 10113781, incorporated herein by reference. Other suitable plant hosts include, but are not limited to, soybean, rapeseed (Brassica napus, B. campestris), pepper, sunflower (Helianthus annus), cotton (Gossypium hirsutum), corn, alfalfa (Medicago sativa), wheat (Triticum sp), barley (Hordeum vulgare), oats (Avena sativa, L), sorghum (Sorghum bicolor), rice (Oryza sativa), cruciferous vegetables (broccoli, cauliflower, cabbage, parsnips, etc.), melons, carrots, celery, parsley, tomatoes, strawberries, peanuts, grapes, grass seed crops, sugar beets, sugar cane, beans, peas, rye, flax, hardwood trees, softwood trees, and forage grasses.

Alternatively, the water-soluble silk proteins may be expressed in mammalian cells such as bovine mammary epithelial alveolar cells or baby hamster kidney cells, as described by Lazaris et al. in Science 295:472-476 (2002), incorporated herein by reference. In both mammalian expression systems, the water-soluble silk proteins were excreted into the culture media. In addition, the silk proteins may be expressed in transgenic animals, which secrete the proteins in their milk or urine, as described by Clark et al. in published US Patent Application No. 2001/0042255 and Karatzas et al. in patent application WO 99/47661, both of which are incorporated herein by reference.

In the preferred embodiment of the present invention, the water-soluble silk proteins are expressed in microbial systems. Suitable microbial expression systems include, but are not limited to, Escherichia, Bacillus, Saccharomyces, Schizosaccharomyces, Pichia, Aspergillus, and Streptomyces. For example, the silk analog proteins DP-1A, DP-1B, and DP-2A may be expressed in E. coli, Bacillus subtilis, or Pichia pastoris, as described by Fahnestock (U.S. Pat. No. 6,268,169). In the most preferred embodiment, the spider silk analog protein DP-2A is expressed in E. coli.

In one embodiment of the present invention, transformed microbial cells, engineered to produce silk proteins or variants thereof, are grown in a suitable growth medium to high density. The growth medium used is not critical. Any conventional medium may be used, including but not limited to, LB medium (containing tryptone, yeast extract, and NaCl), complex media containing organic nitrogen sources such as yeast extract, or minimal or defined media. When the cell growth has reached the desired level, the cells are harvested by means, including but not limited to, centrifugation or filtration. Preferably, the cells are harvested by centrifugation, generally at 2500 to 25,000×g. The cells may be frozen at about −20° C. before proceeding.

The cell paste is suspended in an appropriate buffered aqueous solution, such as a lysis buffer consisting of 50 mM Tris and 5 mM EDTA at pH 7.5. The cells may be disrupted by any convenient means including mechanical, chemical or enzymatic methods. Mechanical methods include, but are not limited to, sonication, homogenation, irradiation, pressing, freeze-thawing or grinding. Chemical methods include, but are not limited to, treatment with alkali such as sodium hydroxide, treatment with detergents such as sodium dodecyl sulfate (SDS) or suspending the cells in a hypotonic solution to induce lysis via osmotic shock. Enzymatic methods include treatment with a lytic enzyme such as lysozyme, although other lytic enzymes are also effective. The preferred cell disruption method is treatment with lysozyme because the silk proteins and variants are shear-sensitive and could be fragmented by physical disruption methods.

DNase I and excess magnesium chloride are added to digest the DNA in the lysate and the lysate is incubated at about 4° C. to about 37° C. for between 15 to 60 min. Optionally, the cell debris may be removed at this time by any convenient means, including but not limited to, centrifugation, filtration, allowing to settle over time, or flocculation using a water soluble polymer such as polyethylene glycol (PEG). The preferred method is centrifugation at about 14,000×g for about 1 h. The cell debris may be washed with an appropriate buffered aqueous solution, such as the lysis buffer described above, to recover any silk protein that may be associated with the debris. The supernatant from this wash is then combined with the supernatant from the cell debris removal step. The pH of the supernatant solution, containing the crude silk protein extract, is adjusted to an acidic pH by the addition of an acid such as acetic acid. Preferably, the pH is adjusted to about 3.0 to 6.0; most preferably the pH is adjusted to about 5.0. This solution may be stored at about 4° C. overnight.

Optionally, any precipitated material may be removed from the extract at this time using any of the methods described above. Preferably, the precipitate is removed by centrifugation at about 14,000×g for about 1 h. The extract is then heated to between about 55° C. to about 100° C., preferably to about 65° C., with gentle stirring for at least 1 min, preferably for at least 10 min, to denature cellular proteins. Optionally, the extract is incubated overnight at about 4° C. Then, the precipitated material is removed by any of the means given above, preferably by centrifugation at about 14,000×g for about 1 h at about 4° C. The temperature of the extract is maintained below about 20° C., preferably between about 0° C. to about 10° C., most preferably at about 0° C. The silk protein is then precipitated at this temperature by the addition of a precipitating agent. Suitable precipitating agents include, but are not limited to, inorganic salts, water-miscible organic solvents such as ethanol, methanol or acetone, or water-soluble organic polymers such as PEG. The preferred precipitation method is the addition of a salt solution, including but not limited to, ammonium sulfate, sodium chloride, sodium sulfate, magnesium sulfate, potassium chloride, calcium chloride, sodium or potassium phosphates. The preferred salt is ammonium sulfate in a saturated solution. The optimum amount of the salt solution added to the extract needs to be determined by routine experimentation because the amount depends upon the particular silk protein being precipitated. It is imperative that this precipitation step be done at a temperature below room temperature (about 20° C.) because it was unexpectedly discovered that the precipitated silk protein obtained under these conditions can be readily redissolved in water. Silk proteins precipitated at room temperature could not be redissolved in water without the addition of harsh chemical reagents. After incubation at the desired temperature for a time sufficient to allow the silk protein to precipitate, typically, between about 5 to about 30 min, the precipitated silk protein is collected by using any of the methods described above, preferably by centrifugation at 14,000×g for about 15 min at about 4° C. The precipitated protein redissolves in water without the use of harsh chemical reagents. The silk protein is then ready for use as appropriate. The precipitated silk protein should not be dried, as this will render the protein insoluble.

In another embodiment of the present invention, the silk protein is excreted by the microbial host into the culture medium. For example, this is the case for silk proteins expressed in Bacillus subtilis, as described by Fahnestock (U.S. Pat. No. 6,268,169). In this case, a modified purification and recovery procedure is used as follows. After growth of the transformed host, the cells are removed from the culture medium using one of the methods described above, preferably by centrifugation. Then the culture media is acidified to a pH between about 3.0 to 6.0; preferably the pH is adjusted to about 5.0, by the addition of an acid, preferably acetic acid is used. Then the purification steps described above are followed from the acidification step.

In another embodiment of the present invention, the silk proteins are expressed in mammalian cells. The silk proteins may be expressed within the cells or secreted into the culture medium. In the former case, the purification and recovery procedure described above for microbial expression within the cells is followed. When the silk protein is excreted by the mammalian cells into the culture medium, the procedure described above for microbial production with excretion into the culture medium is followed. Specifically, the cells are removed from the culture medium and the medium is treated starting at the acidification step.

In another embodiment of the present invention, the silk proteins are produced in transgenic animals. In this case the silk proteins are secreted in the milk or urine of the transgenic animal. The sample comprising the silk protein, including, but not limited to, milk or urine, is treated to remove any solid materials, as described above. The preferred treatment is centrifugation. The sample is then treated as described above starting with the acidification step to purify and recover the silk protein.

In yet another embodiment of the present invention, the silk proteins are expressed in plants. The silk proteins are purified and recovered from the plant tissue by first disrupting the plant tissue, preferably by physical means, including but not limited to, grinding or homogenization. Solid debris is removed as described above, preferably by centrifugation, and the extract is treated as described above starting at the acidification step.

The water-soluble silk proteins of the present invention have applications in compositions for personal care products such as cosmetics, skin care, hair care and hair coloring; in coating of particles, such as pigments; and in bandages to promote wound healing. In these compositions, the water-soluble silk proteins may be used in their native form or they may be modified to form derivatives, which provide a more beneficial effect. For example, the silk protein may be modified by conjugation to a polymer to reduce allergenicity as described by Olsen et al. in U.S. Pat. Nos. 5,981,718 and 5,856,451, both of which are incorporated herein by reference. Suitable modifying polymers include, but are not limited to, polyalkylene oxides, polyvinyl alcohol, poly-carboxylates, poly(vinylpyrolidone), and dextrans. In another example, the water-soluble silk proteins may be modified by selective digestion and splicing of other protein modifiers. For example, the water-soluble silk proteins may be cleaved into smaller peptide units by treatment with acid at an elevated temperature of about 60° C. The useful acids include, but are not limited to, dilute hydrochloric, sulfuric or phosphoric acids. Alternatively, digestion of the water-soluble silk proteins may be done by treatment with a base, such as sodium hydroxide, or enzymatic digestion using a suitable protease may be used. The peptides and proteins may be further modified to provide performance characteristics that are beneficial in specific applications for personal care products. The modification of proteins for use in personal care products is well known in the art. For example, commonly used methods are described by Olsen et al. in U.S. Pat. No. 6,303,752, Weisgerber et al. in U.S. Pat. No. 6,284,246, and by Dietz et al. in U.S. Pat. No. 6,358,501, all of which are incorporated herein by reference. Examples of modifications include, but are not limited to, ethoxylation to promote water-oil emulsion enhancement, siloxylation to provide lipophilic compatibility, and esterification to aid in compatibility with soap and detergent compositions. Additionally, the water-soluble silk proteins may be derivatized with functional groups including, but not limited to, amines, oxiranes, cyanates, carboxylic acid esters, silicone copolyols, siloxane esters, quaternized amine aliphatics, urethanes, polyacrylamides, dicarboxylic acid esters, and halogenated esters. The water-soluble silk proteins may also be derivatized by reaction with diimines and by the formation of metal salts.

Cosmetic and skin care compositions may be anhydrous compositions comprising an effective amount of water-soluble silk protein or derivative thereof in a cosmetically acceptable medium. The uses of these compositions include, but are not limited to, skin care, skin cleansing, make-up, and anti-wrinkle products. An effective amount of a water-soluble silk protein or derivative thereof for cosmetic and skin care compositions is herein defined as a proportion of from about 10⁻⁴ to about 30% by weight, but preferably from about 10⁻³ to 15% by weight, relative to the total weight of the composition. This proportion may vary as a function of the type of cosmetic or skin care composition. Suitable compositions for a cosmetically acceptable medium are described by Philippe in U.S. Pat. No. 6,280,747, incorporated herein by reference. For example, the cosmetically acceptable medium may contain a fatty substance in a proportion generally of from about 10 to about 90% by weight relative to the total weight of the composition, where the fatty phase containing at least one liquid, solid or semi-solid fatty substance. The fatty substance includes, but is not limited to, oils, waxes, gums, and so-called pasty fatty substances. Alternatively, the compositions may be in the form of a stable dispersion such as a water-in-oil or oil-in-water emulsion. Additionally, the compositions may contain one or more conventional cosmetic or dermatological additives or adjuvants, including but not limited to, antioxidants, preserving agents, fillers, surfactants, UVA and/or UVB sunscreens, fragrances, thickeners, wetting agents and anionic, nonionic or amphoteric polymers, and dyes or pigments.

The cosmetic composition may also be in the form of products for nail care, such as a nail varnish. Nail varnishes are herein defined as compositions for the treatment and coloring of nails, comprising an effective amount of water-soluble silk protein or derivative thereof in a cosmetically acceptable medium. An effective amount of a water-soluble silk protein or derivative thereof for use in a nail varnish composition is herein defined as a proportion of from about 10⁻⁴ to about 30% by weight relative to the total weight of the varnish. Components of a cosmetically acceptable medium for nail varnishes are described by Philippe supra. The nail varnish typically contains a solvent and a film forming substance, such as cellulose derivatives, polyvinyl derivatives, acrylic polymers or copolymers, vinyl copolymers and polyester polymers. The composition may also contain an organic or inorganic pigment.

Hair care compositions are herein defined as compositions for the treatment of hair, including but not limited to shampoos, conditioners, lotions, aerosols, gels, and mousses, comprising an effective amount of water-soluble silk protein or derivative thereof in a cosmetically acceptable medium. An effective amount of a water-soluble silk protein or derivative thereof for use in a hair care composition is herein defined as a proportion of from about 10⁻² to about 90% by weight relative to the total weight of the composition. Components of a cosmetically acceptable medium for hair care compositions are described by Philippe supra, and by Omura et al. in U.S. Pat. No. 6,139,851 and Cannell et al. in U.S. Pat. No. 6,013,250, both of which are incorporated herein by reference. For example, these hair care compositions can be aqueous, alcoholic or aqueous-alcoholic solutions, the alcohol preferably being ethanol or isopropanol, in a proportion of from about 1 to about 75% by weight relative to the total weight, for the aqueous-alcoholic solutions. Additionally, the hare care compositions may contain one or more conventional cosmetic or dermatological additives or adjuvants, as given above.

Hair coloring compositions are herein defined as compositions for the coloring, dyeing, or bleaching of hair, comprising an effective amount of water-soluble silk protein or derivative thereof in a cosmetically acceptable medium. An effective amount of a water-soluble silk protein or derivative thereof for use in a hair coloring composition is herein defined as a proportion of from about 104 to about 60% by weight relative to the total weight of the composition. Components of a cosmetically acceptable medium for hair coloring compositions are described by Dias et al., in U.S. Pat. No. 6,398,821 and by Deutz et al., in U.S. Pat. No. 6,129,770, both of which are incorporated herein by reference. For example, hair coloring compositions generally contain a mixture of inorganic peroxygen-based dye oxidizing agent and an oxidizable coloring agent. The peroxygen-based dye oxidizing agent is most commonly hydrogen peroxide. The oxidative hair coloring agents are formed by oxidative coupling of primary intermediates (for example p-phenylenediamines, p-aminophenols, p-diaminopyridines, hydroxyindoles, aminoindoles, aminothymidines, or cyanophenols) with secondary intermediates (for example phenols, resorcinols, m-aminophenols, m-phenylenediamines, naphthols, pyrazolones, hydroxyindoles, catechols or pyrazoles). Additionally, hair coloring compositions may contain oxidizing acids, sequestrants, stabilizers, thickeners, buffers carriers, surfactants, solvents, antioxidants, polymers, non-oxidative dyes and conditioners.

The water-soluble silk proteins and derivatives thereof can also be used to coat pigments and cosmetic particles in order to improve dispersibility of the particles for use in cosmetics and coating compositions. Cosmetic particles are herein defined as particulate materials such as pigments or inert particles that are used in cosmetic compositions. Suitable pigments and cosmetic particles, include, but are not limited to, inorganic color pigments, organic pigments, and inert particles. The inorganic color pigments include, but are not limited to, titanium dioxide, zinc oxide, and oxides of iron, magnesium, cobalt, and aluminum. Organic pigments include, but are not limited to, D&C Red No. 36, D&C Orange No. 17, the calcium lakes of D&C Red Nos. 7, 11, 31 and 34, the barium lake of D&C Red No. 12, the strontium lake D&C Red No. 13, the aluminum lake of FD&C Yellow No. 5 and carbon black particles. Inert particles include, but are not limited to, calcium carbonate, aluminum silicate, calcium silicate, magnesium silicate, mica, talc, barium sulfate, calcium sulfate, powdered Nylon®, perfluorinated alkanes, and other inert plastics.

The effective amount of a water-soluble silk protein or derivative thereof for use in pigment and cosmetic particle coating is herein defined as a proportion of from about 10⁻⁴ to about 50%, but preferably from about 0.25 to about 15% by weight relative to the dry weight of particle. The optimum amount of the water-soluble silk protein or derivative thereof to be used depends on the type of pigment or cosmetic particle being coated. For example, the amount of water-soluble silk protein or derivative thereof used with inorganic color pigments is preferably between about 0.01% and 20% by weight. In the case of organic pigments, the preferred amount of water-soluble silk protein is between about 1% to about 15% by weight, while for inert particles, the preferred amount is between about 0.25% to about 3% by weight. Methods for the preparation of coated pigments and particles are described by Marchi et al. in U.S. Pat. No. 5,643,672, incorporated herein by reference. These methods include: adding an aqueous solution of the water-soluble silk protein or derivative thereof to the particles while tumbling or mixing, forming a slurry of the water-soluble silk protein and the particles and drying, spray drying a solution of the water-soluble silk protein onto the particles or lyophilizing a slurry of the water-soluble silk protein and the particles. These coated pigments and cosmetic particles may be used in cosmetic formulations, paints, inks and the like.

The water-soluble silk proteins and derivatives thereof may also be used as a coating on a bandage to promote wound healing. For this application, the bandage material is coated with an effective amount of the water-soluble silk protein or derivative thereof. For the purpose of a wound-healing bandage, an effective amount of water-soluble silk protein or derivative thereof is herein defined as a proportion of from about 10⁻⁴ to about 30% by weight relative to the weight of the bandage material. The material to be coated may be any soft, biologically inert, porous cloth or fiber. Examples include, but are not limited to, cotton, silk, rayon, acetate, acrylic, polyethylene, polyester, and combinations thereof. The coating of the cloth or fiber may be accomplished by a number of methods known in the art, such as described by Rosenblatt in U.S. patent application Ser. No. 409834, incorporated herein by reference. For example, the material to be coated may be dipped into an aqueous solution containing the water-soluble silk protein or derivative thereof. Alternatively, the solution containing the water-soluble silk protein or derivative thereof may be sprayed onto the surface of the material to be coated using a spray gun. Additionally, the solution containing the water-soluble silk protein or derivative thereof may be coated onto the surface using a roller coat printing process. The wound bandage may include other additives including, but not limited to, disinfectants such as iodine, potassium iodide, povidon iodine, acrinol, hydrogen peroxide, benzalkonium chloride, and chlorohexidine; cure accelerating agents such as allantoin, dibucaine hydrochloride, and chlorophenylamine malate; vasoconstrictor agents such as naphazoline hydrochloride; astringent agents such as zinc oxide; and crust generating agents such as boric acid.

The water-soluble silk proteins of the present invention and derivatives thereof may also be used in the form of a film as a wound dressing material. The use of silk fibroin, in the form of an amorphous film, as a wound dressing material is described by Tsubouchi in U.S. Pat. No. 6,175,053, incorporated herein by reference. The amorphous film comprises a dense and nonporous film of a crystallinity below 10% which contains an effective amount of water-soluble silk protein or derivative thereof. For a film for wound care, an effective amount of water-soluble silk protein or derivative thereof is herein defined as between about 1 to 99% by weight. The film may also contain other components including but not limited to other proteins such as sericin, and desinfectants, cure accelerating agents, vasoconstrictor agents, astringent agents, and crust generating agents, as described above. Other proteins such as sericin may comprise 1 to 99% by weight of the composition. The amount of the other ingredients listed is preferably below a total of about 30% by weight, more preferably between about 0.5 to 20% by weight of the composition. The wound dressing film may be prepared by dissolving the above mentioned materials in an aqueous solution, removing insolubles by filtration or centrifugation, and casting the solution on a smooth solid surface such as an acrylic plate, followed by drying. Due to the solubility of the silk protein purified by the method of this invention, the addition of harsh chemicals is not required. After drying, the film is peeled off the solid surface and is ready for use.

The water-soluble silk proteins of the present invention and derivatives thereof may also be applied to the surface of fibers for subsequent use in textiles. The use of such a protein polymer is unique in this application and provides a monolayer of the protein film on the fiber, resulting in a smooth finish. Ona et al. in U.S. Pat. No. 6,416,558 and Ohashi et al. in U.S. Pat. No. 5,232,611 describe the addition of a finishing coat to fibers. The methods described in these disclosures provide examples of the versatility of finishing the fiber to provide a good feel and a smooth surface. For this application, the fiber is coated with an effective amount of the water-soluble silk protein or derivative thereof. For the purpose of fiber coating for use in textiles, an effective amount of water-soluble silk protein or derivative thereof is herein defined as a proportion of from about 1 to about 99% by weight relative to the weight of the fiber material. The fiber materials include, but are not limited to textile fibers of cotton, polyesters such as rayon and Lycra®, nylon, wool, and other natural fibers including native silk. Compositions suitable for applying the silk protein onto the fiber may include co-solvents such as ethanol, isopropanol, hexafluoranols, isothiocyanouranates, and other polar solvents that can be mixed with water to form solutions or microemulsions. The silk protein-containing solution may be sprayed onto the fiber or the fiber may be dipped into the solution. While not necessary, flash drying of the coated material is preferred. An alternative protocol is to apply the silk protein composition onto woven fibers. An ideal embodiment of this application is the use of water-soluble silk proteins or derivatives thereof to coat stretchable weaves such as used for stockings.

### DESCRIPTION OF PREFERRED EMBODIMENTS

The present invention is a method for the purification and recovery of silk proteins in water-soluble form. In the preferred embodiment, the spider silk protein analog DP-2A (SEQ ID NO:4) was expressed in E. Coli strain FP3276, as described by Fahnestock in U.S. Pat. No. 6,268,169. After the cells reached the desired cell density in the growth medium, the cells were harvested by centrifugation and the cell paste was stored frozen at −20° C. for at least 24 h. The cells were then resuspended in a lysis buffer and disrupted by treatment with lysozyme. The cell suspension was then subjected to two or three freeze-thaw cycles, after which DNA was digested by the addition of DNase I. Cell debris was removed by centrifugation at 14,000×g for 1 h. The cell debris was washed with lysis buffer to recover any entrapped or adsorbed silk protein. The supernatant from the wash was combined with the supernatant from the initial centrifugation and the pH was adjusted to 4.9 using acetic acid. This solution was stored overnight at 4° C. The solution was then centrifuged at 14,000×g for 1 h, incubated at 65° C. for 10 min with gentle stirring, and then incubated overnight at 4° C. The next day the lysate was centrifuged at 14,000×g for 1 h at 4° C. to remove precipitated material. The silk analog protein was then precipitated by the addition of 1/9 volume of a saturated ammonium sulfate solution at 0° C. After a 15 min incubation at 0° C., the precipitated silk analog protein DP-2A was collected by centrifugation at 14,000×g for 15 min at 4° C. The resulting pellet was redissolved in aqueous solution without the addition of harsh chemicals for use in cosmetics, skin care, hair care and hair coloring products; in coating of particles, such as pigments; and in bandages to promote wound healing.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

Unless otherwise specified, standard reagents and solutions used in the following Examples were obtained from Sigma Chemical Co. (St. Louis, Mo.).

The meaning of abbreviations used is as follows “min” means minute(s), “h” means hour(s), “psi” means pounds per square inch, “μL” means microliter(s), “mL” means milliliter(s), “L” means liter(s), “nm” means nanometers, “mM” means millimolar, “M” means molar, “mmol” means millimole(s), “μmole” means micromole(s), “g” means gram(s), “μg” means microgram(s) and “mg” means milligram(s), “g” means the gravitation constant, “A276” means the absorbance at a wavelength of 276 nm, “A260” means the absorbance at a wavelength of 260 nm.

### Example 1

### Purification and Recovery of Spider Silk Analog Protein DP-2A in Soluble Form

The purpose of this Example was to demonstrate the recovery of spider silk analog protein DP-2A in soluble form using a purification method that uses ammonium sulfate fractionation at low temperature.

E. coli strain FP3276 designed for the production of spider silk analog protein DP-2A (SEQ ID NO:4) as described by Fahnestock in U.S. Pat. No. 6,268,169, was cultured as described in Example 5 of U.S. Pat. No. 6,268,169 (incorporated herein by reference) with minor modifications as follows. Strain FP3276 was grown at 36° C. in a BioLafitte fermenter in 10 L of a medium as given in Table 1.

TABLE 1
Composition of Growth Medium Used for Culturing
Host E. coli Strains

| | | |
|---|---|---|
| (NH₄)₂SO₄ | 3.0 | g |
| MgSO₄ | 4.5 | g |
| Na citrate.2H₂O | 0.47 | g |
| FeSO₄.7H₂O | 0.25 | g |
| CaCl₂.2H₂O | 0.5 | g |
| Thiamine-HCl | 0.6 | g |
| Casamino acids | 200 | g |
| Biotin | 0.05 | g |
| K₂HPO₄ | 19.5 | g |
| NaH₂PO₄ | 9.0 | g |
| Glycerol | 100 | g |
| L-Alanine | 10.0 | g |
| Glycine | 10.0 | g |
| Glucose | 200 | g |
| Polypropylene glycol (Aldrich | 10 | mL |
| Chemical Co # 20,233-9) | | |
| ZnSO₄.7H₂O | 0.08 | g |
| CuSO₄.5H₂O | 0.03 | g |
| MnSO₄.H₂O | 0.025 | g |
| H₃BO₃ | 0.0015 | g |
| Ammonium molybdate.4H2O | 0.001 | g |
| CoCl₂.6H₂O | 0.0006 | g |

The fermenter was inoculated with 500 mL overnight culture of FP3276 in 2xYT (16 g Bacto-tryptone, 10 g Bacto-yeast extract, 5 g NaCl per liter at pH 7.0)+2% glucose+50 mg/L kanamycin. The pH was maintained at 6.8 by addition of 40% NH₄OH or 20% H₃PO₄. Dissolved O₂ was maintained at approximately 25%. When the absorption at 600 nm had reached 10-15, production of DP-2A was induced by adding 5 g IPTG (isopropyl β-D-thiogalactopyranoside) contained in 1 L of medium at 1/5 the concentration of original medium in the 10 L tank. After 3 h, the cells were harvested by centrifugation in a GS-3 type rotor in a Sorval Model RC5C refrigerated centrifuge and the cell paste was stored frozen at −20° C. for at least 24 h before proceeding with the purification process.

The cell paste (470 g) was thawed and resuspended in 420 mL of lysis buffer consisting of 50 mM Tris (pH 7.5) and 5 mM EDTA (pH 7.5). Lysozyme was added to the cell suspension to a concentration of 300 μg/mL and the solution was incubated at 4° C. for 1 h. Then, the suspension was quick-frozen in a dry ice-ethanol bath and thawed in a 37° C. bath. This freeze-thaw cycle was repeated. Magnesium chloride and DNase I were added to the solution to give concentrations of 15 mM and 20 μg/mL, respectively. The solution was then incubated at 4° C. for 4.5 h, then an additional 20 μg/mL DNase was added and the suspension incubated at 23° C. for 30 min, until the viscosity had substantially decreased. Cell debris was removed by centrifugation at 14,000×g for 1 h. The cell debris was washed by resuspending it in 450 mL of lysis buffer, followed by centrifugation at 14,000×g for 1 h. The resulting supernatant was combined with the supernatant from the initial centrifugation. The pH of the cleared lysate was adjusted to pH 4.9 with acetic acid. This solution was stored at 4° C. overnight. Half (400 mL) of the lysate (fraction A) was centrifuged at 14,000×g for 1 h, incubated at 65° C. for 10 min with gentle stirring and then, incubated overnight at 4° C. The remaining half (400 mL) of the lysate (fraction B) was incubated at 65° C. for 10 minutes without prior centrifugation. The next day, both lysates A and B received the same treatment separately. The lysates were centrifuged at 14,000×g for 1 h at 4° C. A saturated ammonium sulfate solution (pH 5.0) was added to the supernatants in a volume ratio of 1 to 9. The resulting solutions were incubated on ice for 15 min, and then centrifuged at 14,000×g for 15 min at 4° C. to collect the precipitated DP-2A spider silk analog protein. The resulting pellets were redissolved in water at 4° C. using one tenth the volume of the supernatant. The products were at least 95% pure DP-2A as demonstrated by analysis using the Protein Plus 200 LabChip protocol on the Agilent Technologies (Waldbronn, Germany) model 2100 Bioanalyzer.

The protein products were characterized spectrophotometrically using the empirically determined formulae:

DP-2A (mg/mL)=1.37×A276−0.84×A260; and

Nucleic Acid (mg/mL)=0.06×A260−0.024×A276

The results of the spectrophotometric analysis are given in Table 2.

TABLE 2
Results of the Spectrophotometric Analysis of DP-2A

| Fraction | DP-2A (mg/mL) | Nucleic Acids (mg/mL) |
|---|---|---|
| A | 23.7 | 0.525 |
| B | 20.05 | 1.86 |

### Example 2

### Purification and Recovery of Spider Silk Analog Protein DP-1B in Soluble Form

The purpose of this Example was to demonstrate the recovery of spider silk analog protein DP-1B in soluble form using a purification method that uses ammonium sulfate fractionation at low temperature.

E. coli strain FP3350 designed for the production of spider silk analog protein DP-1B.16 (SEQ ID NO:3), as described by Fahnestock in U.S. Pat. No. 6,268,169, was cultured as described in Example 5 of U.S. Pat. No. 6,268,169 (incorporated herein by reference) with minor modifications as follows. Strain FP3350 was grown at 36° C. in a BioLafitte fermenter in 10 L of a medium as given in Table 1.

The fermenter was inoculated with a 500 mL overnight culture of FP3350 in the same medium with 2% glucose and 50 mg/L kanamycin. The pH was maintained at 6.8 by addition of 40% NH₄OH or 20% H₃PO₄. Dissolved O₂ was maintained at approximately 25%. When the absorption at 600 nm had reached 10-15, production of DP-1 was induced by adding 5 g IPTG contained in 1 liter of medium, ⅕ concentration of original medium in 10 L tank. After 3 h, the cells were harvested by centrifugation in a GS-3 type rotor in a Sorval Model RC5C refrigerated centrifuge and frozen. The yield was 230 g cell paste.

The cell paste was stored frozen at −20° C. for at least 24 h before proceeding with the purification process. The cell paste (230 g) was thawed and resuspended in 230 mL of lysis buffer consisting of 50 mM Tris (pH 7.5) and 5 mM EDTA (pH 7.5). Lysozyme (Sigma Chemical Co., St. Louis, Mo.) was added to the cell suspension to a concentration of 300 μg/mL and the solution was incubated at 4° C. for 1 h. Then, the suspension was quick-frozen in a dry ice-ethanol bath and thawed in a 37° C. bath. This freeze-thaw cycle was repeated. Magnesium chloride and DNase I (Sigma Chemical Co., St. Louis, Mo.) were added to the solution to give concentrations of 15 mM and 20 μg/mL, respectively. The solution was then incubated at 4° C. for 4 h until the viscosity had decreased. Cell debris was removed by centrifugation at 14,000×g for 1 h in a GS-3 type rotor in a Sorval Model RC5C refrigerated centrifuge. The cell debris was washed by resuspending it in 300 mL of lysis buffer, followed by centrifugation at 14,000×g for 1 h. The resulting supernatant was combined with the supernatant from the initial centrifugation. The pH of the cleared lysate was adjusted to pH 4.9 with acetic acid. This solution could be stored at 4° C. overnight. The lysate was centrifuged at 14,000×g for 1 h. The supernatant was incubated at 65° C. for 10 min with gentle stirring and then, incubated overnight at 4° C. The next day, the lysate was centrifuged at 14,000×g for 1 h at 4° C. A saturated ammonium sulfate solution (pH 5.0) was added to the supernatant in a volume ratio of 0.176 to 1. The resulting solution was incubated on ice for 15 min, and then centrifuged at 15,000×g for 15 min at 4° C. to collect the precipitated DP-1B spider silk analog protein. The resulting pellet was redissolved in water at 4° C. using one tenth the volume of the supernatant. Approximately 30% of the protein pellet redissolved.

### Example 3

### Predictive Example of an Alternative Method for the Purification and Recovery of Spider Silk Analog Protein DP-2A in Soluble Form

The purpose of this Example is to demonstrate an alternative method for the recovery of spider silk analog protein DP-2A in soluble form using ammonium sulfate fractionation at low temperature.

E. coli strain FP3276 designed for the production of spider silk analog protein DP-2A (SEQ ID NO:4) is cultured as described in Example 1. The cells are harvested by centrifugation and the cell paste is stored frozen at −20° C. for at least 24 h before proceeding with the purification process. The cell paste is thawed and 300 g is resuspended in 700 mL of lysis buffer consisting of 50 mM Tris (pH 7.5) and 5 mM EDTA (pH 7.5). The resuspended cells are passed through a Manton-Gaulin homogenizer model 15M8TA twice at 10,000 psi. Cell debris is removed by centrifugation at 14,000×g for 1 h. The cell debris is washed by resuspending it in 300 mL of lysis buffer, followed by centrifugation at 14,000×g for 1 h. The resulting supernatant is combined with the supernatant from the initial centrifugation. The pH of the cleared lysate is adjusted to pH 4.9 with acetic acid. This solution can be stored at 4° C. overnight. The lysate is centrifuged at 14,000×g for 1 h, incubated at 65° C. for 10 min with gentle stirring and then, incubated overnight at 4° C. The next day, the lysate is centrifuged at 14,000×g for 1 h at 4° C. A saturated ammonium sulfate solution (pH 5.0 at room temperature) is added to the supernatant in a volume ratio of 1 to 9. The resulting solution is incubated on ice for 15 min, and then centrifuged at 14,000×g for 15 min at 4° C. to collect the precipitated DP-2A spider silk analog protein. The resulting pellet is redissolved in water at 4° C. using one tenth the volume of the supernatant.

### Example 4

### Comparative Example of the Purification and Recovery of Spider Silk Analog Protein DP-2A in Insoluble Form

The purpose of this comparative Example was to demonstrate that the spider silk analog protein DP-2A was obtained in an insoluble form when the ammonium sulfate fractionation was done at room temperature.

The procedure described in Example 1 was used, except that the ammonium sulfate was added at room temperature, approximately 23° C., and the subsequent incubation was also done at room temperature. The resulting DP-2A pellet could not be redissolved in water.

We claim:

1. A method for the purification of water-soluble silk protein from a sample containing water-soluble silk protein comprising;
a) providing a sample comprising silk protein in the presence of contaminating proteins, wherein at least a portion of the silk protein is water soluble;
b) adjusting the pH of the sample of a) to an acidic pH;
c) heating the adjusted sample of b) to a temperature of at least about 55° C.;
d) removing debris from the sample of c);
e) lowering the temperature of the heated sample of (d) to below about 20° C.;
f) adding an effective amount of precipitating agent to the cooled sample of e) for a time sufficient to allow silk protein to precipitate, wherein the precipitated silk protein may be redissolved in an aqueous solution.

2. A method according to claim 1 wherein the pH is adjusted in step b) to pH of about 3.0 to about 6.0.

3. A method according to claim 1 wherein the heating the adjusted extract in step c) is to a temperature of about 55° C. to about 100° C.

4. A method according to claim 1 wherein the temperature is lowered in step e) to from about 0° C. to about 10° C.

5. A method according to claim 1 wherein the precipitating agent is selected from the group consisting of inorganic salts, water-miscible organic solvents, water-soluble organic polymers.

6. A method for the purification of water-soluble silk protein from a host cell containing water-soluble silk protein comprising:
a) providing a host cell comprising silk protein, a portion of which is water soluble;
b) disrupting the host cell to release the silk protein and produce a crude silk extract;
c) adjusting the pH of the crude silk extract to an acidic pH;
d) heating the adjusted extract of c) to a temperature of at least about 55° C.;
e) removing cell debris from the extract of d);
f) lowering the temperature of the heated extract of (e) to below about 20° C.;
g) adding an effective amount of precipitating agent to the cooled extract of f) for a time sufficient to allow silk protein to precipitate, wherein the precipitated silk protein may be redissolved in an aqueous solution.

7. A method according to claim 6 wherein the pH is adjusted in step c) to pH of about 3.0 to about 6.0.

8. A method according to claim 6 wherein the heating the adjusted extract in step d) is to a temperature of about 55° C. to about 100° C.

9. A method according to claim 6 wherein the temperature is lowered in step f) to from about 0° C. to about 10° C.

10. A method according to claim 6 wherein the precipitating agent is selected from the group consisting of inorganic salts, water-miscible organic solvents, water-soluble organic polymers.

11. A method according to claim 6 wherein cell debris is optionally removed after step b).

12. A method according to claim 6 wherein cell debris is optionally removed after step c).

13. A method according to claim 6 wherein at step e) the removal of cell debris is accomplished by centrifugation, filtration, flocculation or settling over time.

14. A method according to claim 6 wherein the host cell is selected from the group consisting of prokaryotic cells, yeasts, fungi, algae, green plants, and mammalian cells.

15. A method according to claim 6 wherein the host cell is disrupted mechanically by a means selected from the group consisting of sonication, irradiation, homogenation, pressing and freeze thawing.

16. A method according to claim 6 wherein the silk protein is selected from the group consisting of the dragline spider silk proteins Spidroin 1 and Spidroin 2, spider silk proteins originating from the minor ampullate gland of Nephila clavipes, and spider silk proteins originating from the flagelliform gland of Nephila clavipes, and variants thereof.

17. A method according to claim 16 wherein the spider silk dragline protein is defined by the formula:
[AGQGGYGGLGXQGAGRGGLGGQGAGAnGG]z
wherein X=S, G or N; n=0-7 and z=1-75, and wherein the value of z determines the number of repeats in the variant protein and wherein the
formula encompasses variations selected from the group consisting of:
(a) when n=0, the sequence encompassing AGRGGLGGQGAGAnGG is deleted;
(b) deletions other than the poly-alanine sequence, limited by the value of n will encompass integral multiples of three consecutive residues;
(c) the deletion of GYG in any repeat is accompanied by deletion of GRG in the same repeat; and
(d) where a first repeat where n=0 is deleted, the
first repeat is preceded by a second repeat where n=6; and wherein the full-length protein is encoded by a gene or genes and wherein said gene or genes are not endogenous to the Nephila clavipes genome.

18. A method according to claim 16 wherein the spider dragline protein has a repeating unit having an amino acid sequence as set forth in SEQ ID NO: 1, SEQ ID NO:2, SEQ ID NO:3 or SEQ ID NO:4.

19. A cosmetic composition, skin care composition, or hair care composition comprising an effective amount of a water-soluble silk protein purified by the method of claim 1.

20. A cosmetic composition, skin care composition, or hair care composition comprising an effective amount of a water-soluble silk protein purified by the method of claim 6.
